(19) United States
(12) Patent Application Publication
(10) Pub. No.: US 2022/0110342 A1
(43) Pub. Date: Apr. 14, 2022

(54) Control of tight junction through the use of polyphenols

(71) Applicant: Guilin Layn Natural Ingredients Corp., Guilin (CN)

(72) Inventors: Juan A. Javierre, Barcelona (ES); Maria Jose Fernandez Llibre, Rochester, MI (US)

(21) Appl. No.: 17/094,318

(22) Filed: Nov. 10, 2020

Related U.S. Application Data
(60) Provisional application No. 63/089,827, filed on Oct. 9, 2020.

(51) Int. Cl. A23K 20/111; A23K 50/20; A23K 50/30; A23K 50/40; A23K 50/75; A23K 50/80; A23K 20/195

(52) U.S. Cl. CPC A23K 20/111; A23K 50/20; A23K 50/30; A23K 20/195; A23K 50/75; A23K 50/80; A23K 50/40

(57) ABSTRACT

A method of controlling a tight junction between epithelial cells of an animal comprises providing the animal with a feed containing polyphenols in an amount effective to increase transmembrane protein levels between the epithelial cells thereby minimizing passage of macromolecules between the epithelial cells of the animal

### CROSS-REFERENCE TO RELATED APPLICATION

The present application is based on and claims the benefit of U.S. provisional patent application Ser. No. 63/089,827, filed Oct. 9, 2020, the content of which is hereby incorporated by reference in its entirety.

### BACKGROUND

This disclosure relates to the use of polyphenols to control the tight junction between and among epithelial cells thereby controlling the passage of harmful macromolecules of microbial origin and/or antigens from the intestinal lumen towards the lamina propria of the epithelium in animals.

Polyphenols are plant-based compounds that are naturally found in fruits, vegetables, herbs, spices and other food sources such as tea, chocolate and wine. Polyphenols act as antioxidants, neutralizing harmful free radicals, and are also thought to reduce inflammation.

It is generally believed that polyphenols, although there are several thousand types of polyphenols, can be categorized in four main groups, flavonoids, phenolic acids, polyphenolic amides, and other polyphenols.

### SUMMARY

This disclosure relates to a method of controlling a tight junction between epithelial cells of an animal. The method comprises providing the animal with a feed containing polyphenols in an amount effective to increase transmembrane protein levels between the epithelial cells thereby minimizing passage of macromolecules between the epithelial cells of the animal.

In a further embodiment, the polyphenols are botanical extracts comprising Vitis vinifera extract, Glycine max extract, Hibiscus sabdariffa extract, Camellia sinensis extract, Ginkgo biloba extract, Coffea arabica extract, Punica granatum extract, Vaccinium myrtillus, Sophora japonica extract or combinations thereof.

In a further embodiment, the polyphenols comprise quercetin, kaempferol, genistin, myricetin, daidzein, chlorogenic acid, caffeic acid, delphidin, cyaniding, epigallocatechin gallate, catechin, epicatechin, gallic acid, ellagic acid, punicalagins, procyanidin B or combinations thereof.

In a further embodiment the animal provided the feed has a monogastric intestinal system.

In a further embodiment, the animal comprises poultry, swine, horses, dogs, cats, or fish.

In a further embodiment, the macromolecules are of microbial origin.

In a further embodiment, the macromolecules are harmful by virtue of an immune response by the animal.

In another embodiment, this disclosure relates to a method of increasing the growth rate of an animal. The method comprises providing the animal with a feed containing polyphenols in an amount effective to increase transmembrane protein levels between the epithelial cells thereby minimizing passage of macromolecules between the epithelial cells of the animal.

Further to this embodiment, the polyphenols are botanical extracts comprising Vitis vinifera extract, Glycine max extract, Hibiscus sabdariffa extract, Camellia sinensis extract, Ginkgo biloba extract, Coffea arabica extract. Punica granaturn extract. Vaccinium myrtillus, Oophora japonica extract or combinations thereof.

Further to this embodiment, the polyphenols comprise quercetin, kaempferol, genistin, myricetin, daidzein, chlorogenic acid, rutin, caffeic acid, delphidin, cyaniding, epigallocatechin gallate, catechin, epicatechin, gallic acid, ellagic acid. punicalagins, procyanidin B or combinations thereof

Further to this embodiment, the animal provided the feed has a monogastric intestinal system.

Further to this, embodiment, the animal comprises poultry, swine, horses, dogs, cats, or fish.

Further to this embodiment, the macromolecules are of microbial origin.

Further to this embodiment, the macromolecules are harmful by virtue of an immune response by the animal.

In yet another embodiment, this disclosure relates to a method of increasing nutrient absorption in an animal. The method comprises providing the animal with a feed containing polyphenols in an amount effective to increase transmembrane protein levels between the epithelial cells thereby minimizing passage of macromolecules between the epithelial cells of the animal.

Further to this embodiment, the polyphenols are botanical extracts Vitis vinifera extract, Glycine max extract, Hibiscus sabdariffa extract, Camellia sinensis extract, Ginkgo Holm. extract, Coffea arabica extract, Punica granatum extract, Vaccinium myrtillus, Sophora japonica extract or combinations thereof,

Further to this embodiment, the polyphenols comprise quercetin, kaempferol, genistin, myricetin, daidzein, chlorogenic acid, rutin, caffeic acid, delphidin, cyaniding, epigallocatechin gallate, catechin, epicatechin, gallic acid, Magic acid, punicalagins, procyanidin 13 or combinations thereof.

Further to this embodiment, the animal provided the feed has a monogastric intestinal system.

Further to this embodiment, the animal comprises poultry, swine, horses, dogs, cats, or fish.

Further to this embodiment, the macromolecules are of microbial origin.

Further to this embodiment, the macromolecules are harmful by virtue of an immune response by the animal.

### DETAILED DESCRIPTION

This disclosure relates to the use of polyphenols to control the tight junction between epithelial cells thereby controlling the passage of harmful macromolecules of microbial origin or antigens of microbial origin from intestinal lumen towards the lamina propria.

For purposes of this disclosure, tight junctions are areas where the membranes of adjacent epithelial cells join together to form a barrier. The cell membranes are connected by strands of transmembrane proteins such as claudins and occludins. The tight junctions form a continuous intercellular barrier within the epithelium controlling the passage of substances from the intestinal lumen towards the lamina propria of the epithelium thereby selectively regulating movement across the epithelium.

The epithelial cells are responsible for taking up nutrients from the gut. Along with this responsibility in taking up nutrients, unwanted substances such as harmful macromolecules of microbial origin need to be controlled or blocked from entering the body through the epithelium. The more that such harmful macromolecules pass through the tight junction, the more the immune system of the animal needs to respond to the numbers of such harmful macromolecules entering the body. This response by the animal then takes up energy which otherwise could be used for positive effects on the animal's welfare which in turn will have an effect on commercial aspects for which the animal is being raised. Such commercial aspects include better growth, muscle mass gain, weight gain, egg production in the case of poultry, and meat quality to name a few. For purposes of this application, the phrase “harmful macromolecules” shall mean such macromolecules that have the herein described detrimental effect on the animal.

The method of this disclosure prevents/minimizes the entry of harmful macromolecules of microbial origin into the animal's body. Thus, the effect of such macromolecules is avoided. This results in the animal's immune system not having to expend energy to fight off the effect of such harmful macromolecules.

The use of certain polyphenols as described further herein controls the pathways through which macromolecules enter the body. Such pathways are referred to as tight junctions which are areas between and among epithelial cells. A network of proteins extend between the epithelial cells. The proteins being affected by the method of this invention primarily include Claudin 1, Claudin 5, Occludin and Zonula Occludens. Chelakkot et al. Experimental & Molecular Medicine (2018) 50:103. The polyphenols of this disclosure increase the abundance, the mass, of such proteins thus forming a tighter barrier type thereby preventing harmful macromolecules from passing through the epithelium.

This disclosure also relates to a method of making a feed supplement and feeding the polyphenol containing supplement to animals. By feeding animals the feed supplement of this disclosure the health and nutritional wellbeing of the animals is either maintained or increased positively by decreasing or eliminating the macromolecules from microbial origin from affecting the animal. The selected polyphenols are plant based and are selected to effectively increase the protein level in the animal's tight junctions of the epithelium layer.

The method of this disclosure can also be used as a treatment for leaky gut syndrome. By the phrase, leaky gut syndrome, as used herein, it is meant the condition in which the small intestine lining becomes damaged, allowing the macromolecules to pass between and among epithelial cells, resulting in cell damage or inflammation of the intestine. Once the macromolecules pass between cells, the animal's immune system recognizes them and triggers an immune response to destroy and remove the harmful macromolecules. This inflammatory process and immune system activation may consume significant amounts of energy and nutrients. While an appropriate inflammatory response is necessary, excessive or prolonged inflammation can become detrimental to the animal. Animals that suffer from chronic, prolonged inflammation may be unable to mount a successful immune response in the future. Chronic inflammation pulls nutrients and energy away from other key functions within the animal such as growth, reproduction, and meat or egg production.

The preferred animals for the polyphenol containing feed have a monogastric intestinal system and include without limitation poultry such as chickens, swine, horses, pets such as dogs and cats, and fish. Since the polyphenols of this disclosure are plant based such feed or feed supplements can be considered and promoted as natural and therefore healthy supplements for animals.

In one embodiment, the feed of this disclosure is a blend of polyphenol-rich botanical extracts, including but not limited to Vitis vinifera extract, Glycine max extract, Hibiscus sabdariffa extract, Camellia sinensis extract, Ginkgo biloba extract, Coffea arabica extract, Punica granatum extract, Vaccinium myrtillus, or Sophora japonica extract. The blend of polyphenol extracts may be any of the aforesaid alone or in combination.

The polyphenols obtained from said extracts include, but are not limited to quercetin, kaempferol, genistin, myricetin, daidzein, chlorogenic acid, caffeic acid, rutin, delphidin, cyaniding, epigallocatechin gallate, catechin, epicatechin, gallic acid, ellagic acid, punicalagins, procyanidin B or combinations thereof.

The blend of polyphenols is added continuously to the diet of the intended target animal; alternatively, they can also be administered mixed with drinking water.

### EXAMPLE

In a trial involving about 2,000 broilers, the feed of this disclosure was fed ad libitum for 6 weeks. Results from trials using the polyphenol blend on broiler chickens are described in the tables below, Table 1 below shows the relative abundance of Tight Junction proteins comparing a control group with a group of broilers fed the polyphenol feed of this disclosure.

TABLE 1
Relative abundance of Tight Junction proteins.
Percent increase of polyphenol-treated
birds over the untreated controls

| | | | | Zonula |
|---|---|---|---|---|
| | Claudin1 | Claudin5 | Occludin | Occludens |
| Control | 100 | 100 | 100 | 100 |
| PF | 359 | 188 | 237 | 160 |

Table 2 below shows the comparative weight gain of broilers fed the polyphenols of this disclosure in comparison to a control group of broilers. The numerical values in Table 2 are mean values for both the control and the polyphenol fed group of broilers. Measurements were done weekly from week 1 to week 6. 0 is day one of the first week, the day the trial started.

TABLE 2
Body weight of broilers in grams. Feed provided ad libitum

| Week | 0 | 1 | 2 | 3 | 4 | 5 | 6 |
|---|---|---|---|---|---|---|---|
| Control | 43.8 | 154 | 359 | 720 | 1237 | 1797 | 2361 |
| Group | | | | | | | |
| Polyphenol | 43.2 | 161 | 379 | 755 | 1322 | 1902 | 2569 |
| Feed | | | | | | | |
| Group | | | | | | | |

Table 3 below shows feed conversion ratio (FCR), which is a measure of efficiency. (Feed eaten (kg)/body mass gain (kg))

TABLE 3
Cumulative FCR on a weekly basis

| Week | 1 | 2 | 3 | 4 | 5 | 6 |
|---|---|---|---|---|---|---|
| Control | 0.819 | 1.301 | 1.401 | 1.493 | 1.640 | 1.732 |
| Group | | | | | | |
| Polyphenol | 0.786 | 1.265 | 1.351 | 1.437 | 1.576 | 1.707 |
| Feed | | | | | | |
| Group | | | | | | |

1. A method of controlling a tight junction between epithelial cells of an animal, the method comprising:
providing the animal with a feed containing polyphenols in an amount effective to increase transmembrane protein levels between the epithelial cells thereby minimizing passage of macromolecules between the epithelial cells of the animal.

2. The method of claim 1 wherein the polyphenols are botanical extracts comprising Vitis vinifera extract, Glycine max extract, Hibiscus sabdariffa extract, Camellia sinensis extract, Ginkgo biloba extract, Coffea arabica extract, Punica granatum extract, Vaccinium myrtillus, Sophora japonica extract, or combinations thereof.

3. The method of claim 1 wherein the polyphenols comprise quercetin, kaempferol, genistin, myricetin, daidzein, chlorogenic acid, rutin, delphidin, epigallocatechin gallate, procyanidin B., or combinations thereof.

4. The method of claim 1 wherein the animal provided the feed has a monogastric intestinal system.

5. The method of claim 4 wherein the animal comprises poultry, swine, horses, dogs, cats, or fish.

6. The method of claim 1 wherein the macromolecules are of microbial origin.

7. The method of claim 1 wherein the macromolecules are harmful by virtue of an immune response by the animal.

8. A method of increasing growth rate of an animal, the method comprising:
providing the animal with a feed containing polyphenols in an amount effective to increase transmembrane protein levels between the epithelial cells thereby minimizing passage of macromolecules between the epithelial cells of the animal.

9. The method of claim 8 wherein the polyphenols are botanical extracts comprising Vitis vinifera extract, Glycine max extract, Hibiscus sabdariffa extract, Camellia sinensis extract, Ginkgo biloba extract, Coffea arabica extract, Punica granatum extract, Vacciniurn myrtillus, Sophora japonica extract, or combinations thereof.

10. The method of claim 8 wherein the polypenols comprise quercetin, kaempferol, genistin, daidzein, chlorogenic acid, rutin, delphidin, epigallocatechin gallate, procyanidin B., or combinations thereof.

11. The method of claim 8 wherein the animal provided the feed has a mogastric intestinal system.

12. The method of claim 11 wherein the animal comprises poultry, swine, horses, dogs, cats, or fish.

13. The method of claim 8 wherein the macromolecules are of microbial origin.

14. The method of claim 8 wherein the macromolecules are harmful by virtue of an immune response by the animal.

15. A method of increasing nutrient absorption in an animal, the method comprising:
providing the animal with a feed containing polyphenols in an amount effective to increase transmembrane protein levels between the epithelial cells thereby minimizing passage of macromolecules between the epithelial cells of the animal.

16. The method of claim 15 wherein the polyphenols are botanical extracts comprising Vitis vinifera extract, Glycine max extract, Hibiscus sabdariffa extract, Carnellia sinensis extract, Ginkgo biloba extract, Coffea arabica extract, Tunica granatum. extract, Vaccinium myrtillus, Sophora japonica extract, or combinations thereof.

17. The method of claim 15 wherein the polypenols comprise quercetin, kaempferol, genistin, myricetin, daidzein, chlorogenic acid, rutin, delphidin, epigallocatechin gallate, procyanidin B., or combinations thereof.

18. The method of claim 15 wherein the animal provided the feed has a mogastric intestinal system.

19. The method of claim 18 wherein the animal comprises poultry, swine, horses, dogs, cats, or fish.

20. The method of claim 15 wherein the macromolecules are of microbial origin.

21. The method of claim 15 wherein the macromolecules are harmful by virtue to an immune response by the animal.
